(19) United States
(12) Patent Application Publication
(10) Pub. No.: US 2022/0241441 A1
(43) Pub. Date: Aug. 4, 2022

(54) UVC Shopping Cart Cleaner

(71) Applicant: Scott Hickey, Vista, CA (US)

(72) Inventor: Scott Hickey, Vista, CA (US)

(21) Appl. No.: 17/163,500

(22) Filed: Jan. 31, 2021

(51) Int. Cl. A61L 2/10

(52) U.S. Cl. CPC A61L 2/10; A61L 2202/11; A61L 2202/121; A61L 2202/122; A61L 2202/14

(57) ABSTRACT

A system and method for providing automated UVC cleaning of shopping carts is disclosed. The system includes a first enclosing tunnel for containing one or more shopping carts during cleaning, one or more automatically opening and closing doors for enclosing any access to the first enclosing tunnel, a door lock coupled to each of the automatically opening and closing doors, a conveyor subsystem for loading and unloading shopping carts for cleaning within the first enclosing tunnel, and one or more UVC light source emitters contained within the first enclosing tunnel.

### TECHNICAL FIELD

This application relates in general to a system and method for providing a cleaning aid, and more specifically, to a system and method for providing automated UVC cleaning of shopping carts.

### BACKGROUND

Typically shopping carts were not cleaned prior to Covid-19; if these carts were cleaned, a company would pressure wash them on occasion. Now, an employee is assigned to hand wipe carts with liquid cleaners which exposes the employee to possible infection. Failure to adequately and effectively clean shopping carts after each use puts both customers and employees at risk of exposure to bacteria and viruses. A safe, automated, and hands-free cleaning system is not readily available.

Therefore, a need exists for a system and method for providing automated UVC cleaning of shopping carts. The present invention attempts to address the limitations and deficiencies in prior solutions according to the principles and example embodiments disclosed herein.

### SUMMARY

In accordance with the present invention, the above and other problems are solved by providing a system and method for providing automated UVC cleaning of shopping carts according to the principles and example embodiments disclosed herein.

In one embodiment, the present invention is a system and method for providing automated UVC cleaning of shopping carts. The system includes a first enclosing tunnel for containing one or more shopping carts during cleaning, one or more automatically opening and closing doors for enclosing any access to the first enclosing tunnel, a door lock coupled to each of the automatically opening and closing doors, a conveyor subsystem for loading and unloading shopping carts for cleaning within the first enclosing tunnel, and one or more UVC light source emitters contained within the first enclosing tunnel.

In another aspect of the present disclosure, the system further includes a control panel for operating the system. The control panel has a controller-timer for activating the one or more automatically opening and closing doors, the door locks coupled to each of the one or more automatically opening and closing doors, the conveyor subsystem, and the UVC light source emitters, an input keypad, and an activating key for insertion into a control panel keyhole.

In another aspect of the present disclosure, the controller-time includes a memory having instructions stored thereon, and a processor configured to execute the instructions on the memory to cause the control panel to receive an activation input when the activating key rotates within the control panel keyhole, close the one or more automatically opening and closing doors, activate the UVC light source emitters, after the UVC light source emitters have been active for a predetermined length of time, deactivate the UVC light source emitters, and open the one or more automatically opening and closing doors.

In another aspect of the present disclosure, the control panel lock the door locks on the one or more automatically opening and closing doors after the doors have been closed, activates the UVC light source emitters only when the door locks remain locked, and unlocks the door locks to open the one or more automatically opening and closing doors only when the UVC light source emitters are deactivated.

In another aspect of the present disclosure, the control panel further detects a presence of shopping carts within the enclosing tunnel, determines whether the one or more automatically opening and closing doors are open, unlocks and opens the one or more automatically opening and closing doors, when the one or more automatically opening and closing doors are closed, activates the conveyor subsystem to push the shopping carts out of the enclosing tunnel when the shopping carts are detected and the one or more automatically opening and closing doors are open, detect when a last shopping cart has exited the enclosing tunnel, and deactivate the conveyor subsystem.

In another aspect of the present disclosure, the control panel further detects a jam of one or more shopping carts within the enclosing tunnel, activates an alarm on the control panel, and deactivates the conveyor subsystem.

In another aspect of the present disclosure, the control panel further detects a presence of shopping carts within the enclosing tunnel. When no shopping carts are detected within the enclosing tunnel, the control panel further detect a presence of shopping carts within an entrance to the enclosing tunnel, determine whether the one or more automatically opening and closing doors are open, unlock and open the one or more automatically opening and closing doors, when the one or more automatically opening and closing doors are closed, activates the conveyor subsystem to push the shopping carts into the enclosing tunnel, detects when a last shopping cart has exited the entrance to the enclosing tunnel, deactivates the conveyor subsystem, and closes and locks the one or more automatically opening and closing doors

In another aspect of the present disclosure, the system further includes a second enclosing tunnel coupled to the first enclosing tunnel, a second conveyor subsystem for loading and unloading shopping carts for cleaning within the second enclosing tunnel, the second conveyor subsystem receives shopping carts from the first conveyor subsystem and pushes the shopping carts out of the second enclosing tunnel, and one or more UVC light source emitters contained within the second enclosing tunnel.

In another aspect of the present disclosure, the control panel activates the first conveyor subsystem and the second conveyor subsystem at the same time, deactivates the first conveyor subsystem and the second conveyor subsystem at the same time, activates the UVC light source emitters within the first enclosing tunnel and the UVC light source emitters within the second enclosing tunnel at the same time, and deactivates the UVC light source emitters within the first enclosing tunnel and the UVC light source emitters within the second enclosing tunnel at the same time.

In another aspect of the present disclosure, the system has only one automatically opening and closing door and the conveyor subsystem pushes the shopping carts into the enclosing tunnel and out of the enclosing tunnel using the same opening.

In another embodiment, the present invention comprises a method for providing automated UVC cleaner unit of shopping carts, the unit comprises a first enclosing tunnel for containing one or more shopping carts during cleaning, one or more automatically opening and closing doors for enclosing any access to the first enclosing tunnel, a door lock coupled to each of the automatically opening and closing doors, a conveyor subsystem for loading and unloading shopping carts for cleaning within the first enclosing tunnel, and one or more UVC light source emitters contained within the first enclosing tunnel. The method receives an activation input when the activating key rotates within the control panel keyhole, closes the one or more automatically opening and closing doors, activates the UVC light source emitters, after the UVC light source emitters have been active for a predetermined length of time, deactivates the UVC light source emitters, and opening the one or more automatically opening and closing doors.

In another aspect of the present disclosure, the method further locks the door locks on the one or more automatically opening and closing doors after the doors have been closed, activates the UVC light source emitters only when the door locks remain locked, and unlocks the door locks to open the one or more automatically opening and closing doors only when the UVC light source emitters are deactivated.

In another aspect of the present disclosure, the method further detects a presence of shopping carts within the enclosing tunnel, determines whether the one or more automatically opening and closing doors are open, unlocks and opens the door locks coupled to each of the one or more automatically opening and closing doors, when the one or more automatically opening and closing doors are closed, activates the conveyor subsystem to push the shopping carts out of the enclosing tunnel when the shopping carts are detected and the one or more automatically opening and closing doors are open, detects when a last shopping cart has exited the enclosing tunnel, and deactivates the conveyor subsystem.

In another aspect of the present disclosure, the method further detects a presence of shopping carts within an entrance to the enclosing tunnel, determines whether the one or more automatically opening and closing doors are open, unlocks and opens the door locks coupled to each of the one or more automatically opening and closing doors, when the one or more automatically opening and closing doors are closed, activates the conveyor subsystem to push the shopping carts into the enclosing tunnel, detects when a last shopping cart has exited the entrance to the enclosing tunnel, deactivates the conveyor subsystem, and closes and locks the door locks coupled to each of the one or more automatically opening and closing doors.

In another aspect of the present disclosure, the method further detects a jam of one or more shopping carts within the enclosing tunnel, activates an alarm on the control panel. and deactivates the conveyor subsystem.

The foregoing has outlined rather broadly the features and technical advantages of the present invention in order that the detailed description of the invention that follows may be better understood. Additional features and advantages of the invention will be described hereinafter that form the subject of the claims of the invention.

It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features that are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only, and is not intended as a definition of the limits of the present invention.

### BRIEF DESCRIPTION OF THE DRAWINGS

Referring now to the drawings in which like reference numbers represent corresponding parts throughout:

FIG. 1 illustrates an example embodiment of system and method for providing automated UVC cleaning of shopping carts according to the present invention.

FIGS. 2a-c illustrate another example embodiment of a system and method for providing automated UVC cleaning of shopping carts according to the present invention.

FIG. 3 illustrates a block diagram of the components within a system and method for providing automated UVC cleaning of shopping carts according to the present invention.

FIG. 4 illustrates a flowchart of a sequence of steps to implement a method for providing automated UVC cleaning of shopping carts according to the present invention.

FIG. 5 illustrates a flowchart of a sequence of steps to implement a method for providing automated UVC cleaning of shopping carts according to the present invention.

FIG. 6 illustrates a flowchart of a sequence of steps to implement a method for loading and unloading shopping carts by an automated UVC cleaning of shopping carts according to the present invention.

### DETAILED DESCRIPTION

This application relates in general to a system and method for providing a cleaning aid, and more specifically, to a system and method for providing automated UVC cleaning of shopping carts according to the present invention.

Various embodiments of the present invention will be described in detail with reference to the drawings, wherein like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

In describing embodiments of the present invention, the following terminology will be used. The singular forms “a,” “an,” and “the” include plural referents unless the context clearly dictates otherwise. Thus, for example, reference to “a needle” includes reference to one or more of such needles and “etching” includes one or more of such steps. As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. As used herein, the singular forms “a,” “an,” and “the” are intended to include the plural forms as well, unless the context clearly indicates otherwise.

It further will be understood that the terms “comprises,” “comprising,” “includes,” and “including” specify the presence of stated features, steps or components, but do not preclude the presence or addition of one or more other features, steps or components. It also should be noted that in some alternative implementations, the functions and acts noted may occur out of the order noted in the figures. For example, two figures shown in succession may in fact be executed substantially concurrently or may sometimes be executed in the reverse order, depending upon the functionality and acts involved.

The terms “worker,” and “user” refer to an entity, e.g. a human, using a system and method for providing automated UVC cleaning of shopping carts associated with the invention. The term user herein refers to one or more users.

The term “invention” or “present invention” refers to the invention being applied for via the patent application with the title “UVC CleanzCart.” Invention may be used interchangeably with cleaner.

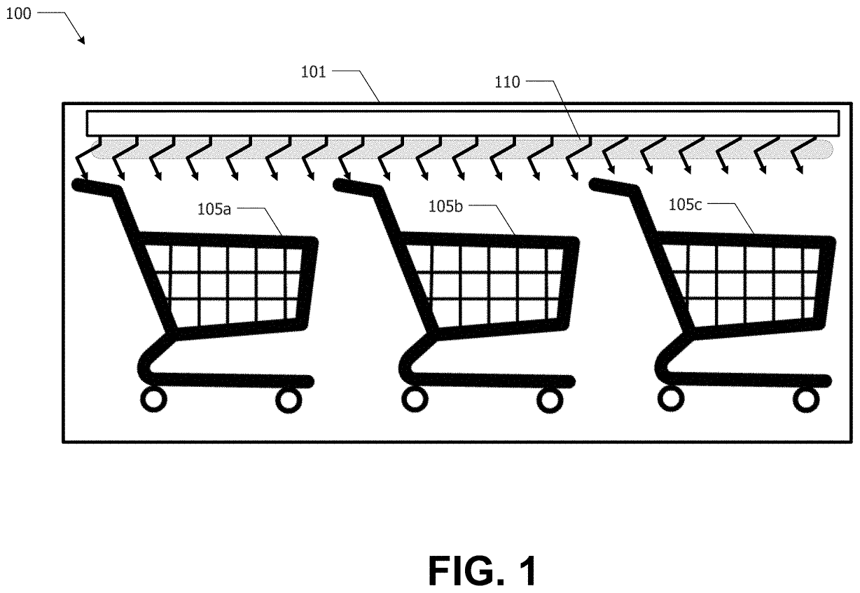

In general, the present disclosure relates to a system and method for providing a cleaning aid. To better understand the present invention, FIG. 1 illustrates an example embodiment of a system and method for providing automated UVC cleaning of shopping carts according to the present invention.

The automated UVC cart cleaner 100 is an automated machine that disinfects shopping carts 105a-c. The automated UVC cart cleaner 100 has a modular design allowing multiple units to be fastened together to accommodate an amount of carts to be cleaned per hour needed at a particular location.

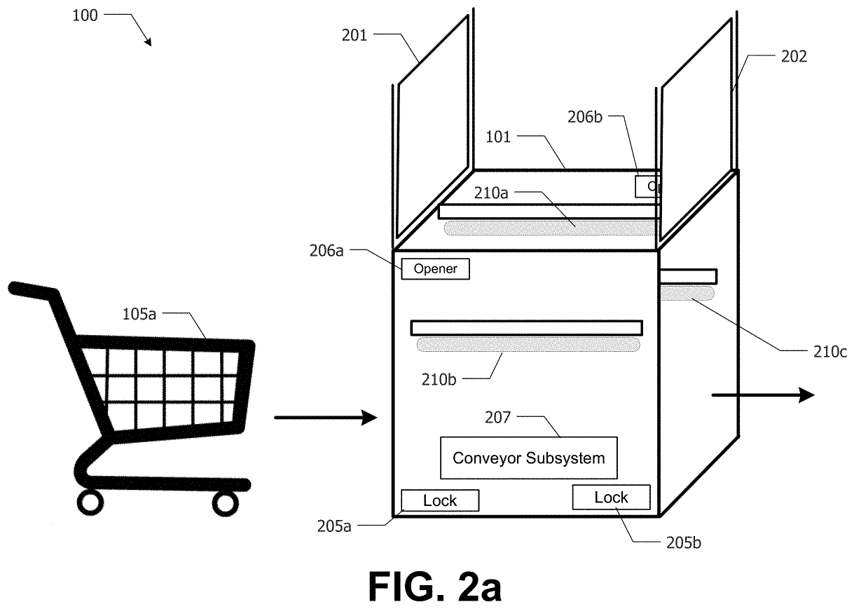

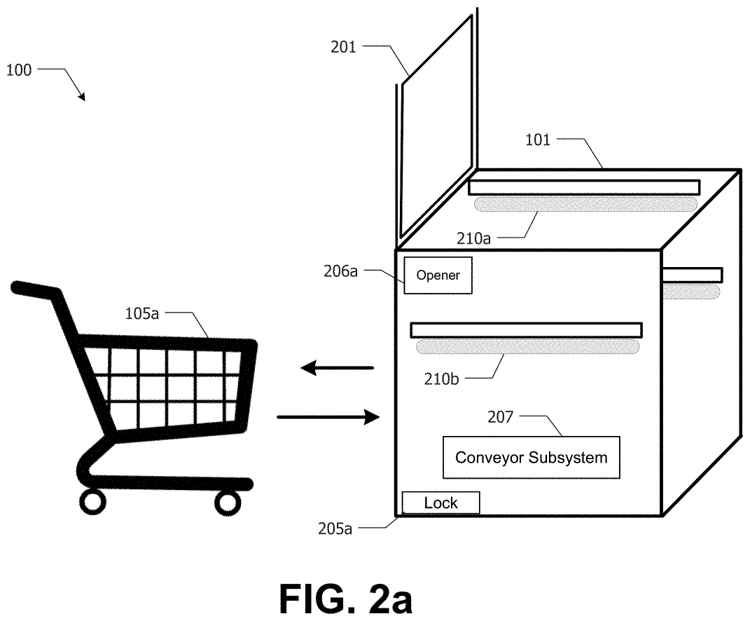

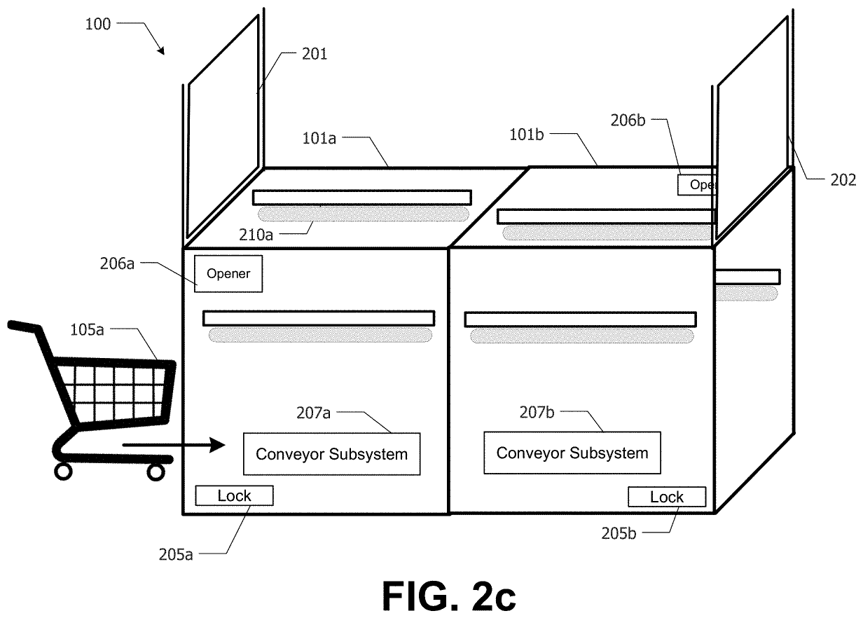

The automated UVC cart cleaner 100 is made from welded steel tubing, approximately 96″ long, 55″ tall, and a width depending on the style and size of cart to be cleaned. In a preferred embodiment, the automated UVC cart cleaner 100 is 35″ wide to accommodate the widest cart currently on the market. The modular design of the automated UVC cart cleaner 100 allows a variety of retail stores with shopping carts 105a-c to reconfigure the automated UVC cart cleaner 100 to accommodate their cleaning needs. The automated UVC cart cleaner 100 comprises a tunnel 101 with automated doors on each end as shown in FIG. 2. One end of the automated UVC cart cleaner 100 is “enter” and the opposite end is “exit.”

The automated UVC cart cleaner 100 may be modified to use only one door 201 for stores with space restraints. The shopping carts 105a-c are loaded in one end of the automated UVC cart cleaner 100 and removed from the same end. Also, multiple automated UVC cart cleaner units 100 may be fastened together to increase the number of carts to be disinfected per hour.

The automated UVC cart cleaner 100 accommodates different types and styles of shopping carts. Three basic variations of base the automated UVC cart cleaner unit 100 include:

1. Two door tunnel, approximately 96″ long;

2. Two door tunnel, multiple modules, approximately 192″ (2 modules); and

3. Two door tunnel, multiple modules, approximately 258″ (3 modules), etc.

Within the automated UVC cart cleaner 100, one or more banks of UVC light source emitters 110 are placed about the inner surfaces of the tunnel to illuminate all parts of the shopping carts 105a-c with the UVC light. An example UVC light source emitter 110 delivers 1μ Watts/cm² to 5μ Watts/cm² per inch of glass measured at 1 m (3 ft), when tested in a 500 fpm (2.54 mis) airstream of 55° F. (13° C.). This output of conventional UVC light source emitters 110 is typical to ensure the delivery of sufficient germicidal energy to produce a desired result. Additionally, automated door openers 206a-b and a conveyor 207 or cart push mechanism (not shown) also may be included within the automated UVC cart cleaner 100.

FIG. 2 illustrates another example embodiment of a system and method for providing automated UVC cleaning of shopping carts according to the present invention. FIG. 2a shows and an example of a one-unit arrangement of the automated UVC cart cleaner 100 according to the present invention. With the automated UVC cart cleaner 100 operating, an employee activates a control panel 310 and the entrance opener 206a opens an entrance door 201, the employee loads a plurality of nested carts 105a-c into the automated UVC cart cleaner 100, and the entrance door 201 automatically closes using the entrance opener 206a and locks 205a-b.

In a preferred embodiment, a set of three separate banks of UVC light source emitters 110 is activated for a set duration of time which disinfects the shopping carts 105a-c. To provide coverage throughout the entire automated UVC cart cleaner 100, a back of four UVC light source emitters 110a are located along a top inside surface of the automated UVC cart cleaner 100, and a second and third UVS light source emitters 201b-c are located on each of the side walls of the automated UVC cart cleaner 100. The interior of the automated UVC cart cleaner 100 is polished aluminum for optimum reflection, bulbs covered with metal grid mesh to protect from breakage as shown in FIG. 4.

After the preset amount of time has passed, the exit door 202 is unlocked, the UVC light source emitters 110 are deactivated, and the exit door 202 is automatically opened. Once the exit door 202 is opened by the exit opener 206b, a screw drive conveyor subsystem 207 pushes the group of nested carts out of the exit for an employee to disperse to customers.

FIG. 2b shows and an example of a one-door arrangement of the automated UVC cart cleaner 100 according to the present invention. In this embodiment, a single automated UVC cart cleaner 100 tunnel 101 is shown having a single automated door blocking tube mounting angle 201 on one side. The shopping carts 105a-c are loaded into the open door 201 by the conveyor subsystem 207 and the door 201 closes and locks. The UVC light source emitters 110 are activated and when the cleaning cycle ends, the shopping carts 105a-c are automatically pushed out of the single door blocking tube mounting angle 201 by the conveyor subsystem 207 once the door has been opened.

FIG. 2c shows and an example of a multi-unit arrangement of the automated UVC cart cleaner 100 according to the present invention. In this alternate embodiment, two automated UVC cart cleaner 100 units 101a-b are concatenated together to accept a larger number of shopping carts 105a for cleaning in a single cycle. A pair of conveyor subsystems 207a-b work together to automatically move the shopping carts 105a-c into and through the first of the two automated UVC cart cleaner 100 units 101a and into the second automated UVC cart cleaner 100 unit 101. The pair of conveyor subsystems 207a-b are arranged to permit the shopping carts to pass out of the first automated UVC cart cleaner 100 unit 101a and engage the second conveyor subsystem 207b within the second automated UVC cart cleaner 100 unit 101b. The door 201 on the first automated UVC cart cleaner 100 unit 101a acts as the entry door and the door 202 on the second automated UVC cart cleaner 100 unit 101b acts as the exit door.

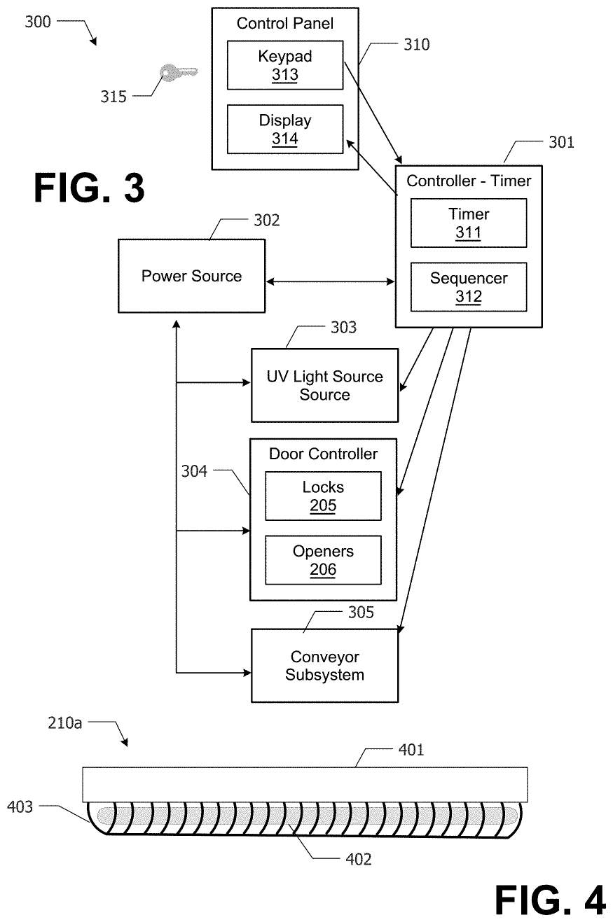

FIG. 3 illustrates a block diagram of the components within a system and method for providing automated UVC cleaning of shopping carts according to the present invention. The automated UVC cart cleaner 100 includes a set of components: a controller-timer 301, a power source 302, one or more UVC light source emitters 303, a door controller 304, a conveyor subsystem 305, and a user control panel 310. The controller-timer 301 generates control signals to instruct the other components of the system to perform their relevant functions. The controller-timer 301 is electrically coupled to the control panel 310 to provide an operator with operational control over the automated UVC cart cleaner 100.

The controller timer 301 includes a timer 311 to measure a set time period during which the UVC light source emitters 210 are activated to illuminate shopping carts 105a-c within the automated UVC cart cleaner 100. The controller timer 301 also includes a sequencer 312 to automate the loading of the shopping carts 105a-c, opening and closing of the doors 201-202, activating and deactivating the door locks 205, activating the UVC light source emitters 110, and interrupting any operation upon detecting an error or failure. The sequencer 312 may be constructed using one or more timers and combinatorial logic, a digital electronic state machine, a programmable processing device, and a set of software-based instructions. The sequencer 312 implements the instructions to implement the steps of the method of FIG. 4 below.

The power source 302 provides electrical power used by the other components to operate. The power source 302 receives ordinary AC current from a connection to the internal power available at the retail location. This input current is typically 120/240 V AC power provided by local utilities. The power source 302 converts the incoming AC voltage to one or more voltages used by these various components as needed. The power source 302 may also condition and filter the incoming AC voltages to reduce random voltage spikes that may occur from time to time. The power source 302 may include an emergency stop (not shown) that disconnects power to the internal devices using either an error signal from the controller timer 301 or an emergency stop button depressed by a user.

The one or more UVC light source emitters 303 provide the UV energy to disinfect the shopping carts 105a-c from many pathogens, fungus, bacteria, viruses, and the like. The UVC light source emitters 303 are placed in one or more locations, typically on at least two sides of the tunnel, that provide UVC light illumination on a significant portion of the exposed surfaces of the shopping carts 105a-c. The UVC light source emitters 303 are configured to minimize as much as possible any parts of the shopping carts 105a-b that may be shadowed by the nesting of the carts before insertion into the automated UVC cart cleaner 100.

As noted above, an example UVC light source emitter 110 delivers 1μ Watts/cm² to 5μ Watts/cm² per inch of glass measured at 1 m (3 ft), when tested in a 500 fpm (2.54 mis) airstream of 55° F. (13° C.). This output of conventional UVC light 110 is typical for these emitters to ensure the delivery of sufficient germicidal energy to produce a desired result.

The door controller 304 connects to and operates door locks 205a-b on the entrance door 201 and the exit door 202. The door controller 304 activates the locks 205a-b and provides lock state signals to the controller-timer 301 to inform the controller-timer 301 whenever one or more of the doors 201-202 is opened. The door controller 304 also connects to and operates the door openers 206a-b that extend to open and retract to close the doors 201-202 as needed. The doors in a preferred embodiment open upward and ay be raised using a pushrod or similar device that raises the door when the pushrod extends. Other door and door opening arrangements including many garage door type openers may also be used.

The conveyor subsystem 305 engages the shopping carts 105a-c as they enter the automated UVC cart cleaner 100 and automatically move the one or more nested carts into the automated UVC cart cleaner 100 to permit the doors 201-202 to close and lock. the conveyor subsystem 305 may include an optical contact sensor (not shown) to indicate when a shopping cart has entered into the automated UVC cart cleaner 100 and is in a position to engage with the conveyor subsystem 305. The optical contact sensor may also detect when a last shopping cart has completely entered the automated UVC cart cleaner 100 allowing the doors 201-202 to close and lock. The conveyor subsystem 305 operates until the last shopping cart 105a-c has exited the automated UVC cart cleaner 100. When more than one automated UVC cart cleaner unit 100 are connected to create a larger cleaner, a shopping cart 105a-c engages with the conveyor subsystem 305 in the next unit in the sequence before the cart disengages from the conveyor subsystem 305 in the first unit.

The user control panel 310 provides an operator with an input device 313 to enter operating commands into the controller-timer 301 as well as provides output devices 314 to provide the operator with state or status information regarding the automated UVC cart cleaner 100 and its operation. The user control panel 310 may include one or more switches (not shown) to provide an electrical signal to the timer-controller 301 to start and stop the operation of the conveyor subsystem 305, the door controller 304, the UVC light source emitters 303, and the power supply 302. The user control panel 310 also may include one or more status and error lights, LEDs, and visual indicators (not shown) to provide useful information to the operator regarding the state of operation of the automated UVC cart cleaner 100. The user control panel 310 may also include a key 315 to be used by the operator to activate and deactivate the device 100. Use of the key assists to prevent unauthorized and unintentional activation of the components if the automated UVC cart cleaner 100, including the automated doors blocking tube mounting angle 201-locking pin 202, the UVC lights 110, and the conveyor subsystem 305. The user control panel 310, in other embodiments may be constructed using a computer display device 314 and a keypad 313 or a touchscreen display device, among other user interface mechanisms.

FIG. 4 illustrates an example UVC lamp fixture for use within an automated UVC cleaning of shopping carts according to the present invention. Each of the banks of UVC light source emitters 210a include 4 separate UVC light source emitters 402 coupled into electrical fixtures within a base unit 401. Each of the 4 separate UVC light source emitters 402 and enclosed within a protective wire mesh 403 to protect the emitters from brakeage when shopping carts are loaded and unloaded. All of the light source emitters 402 within the back of emitters 210a are activated at the same time for the same time duration. The three packs of UVC light source emitters 201a-c are also activated at the same time for the same duration during the cleaning cycle of the automated UVC cart cleaner 100.

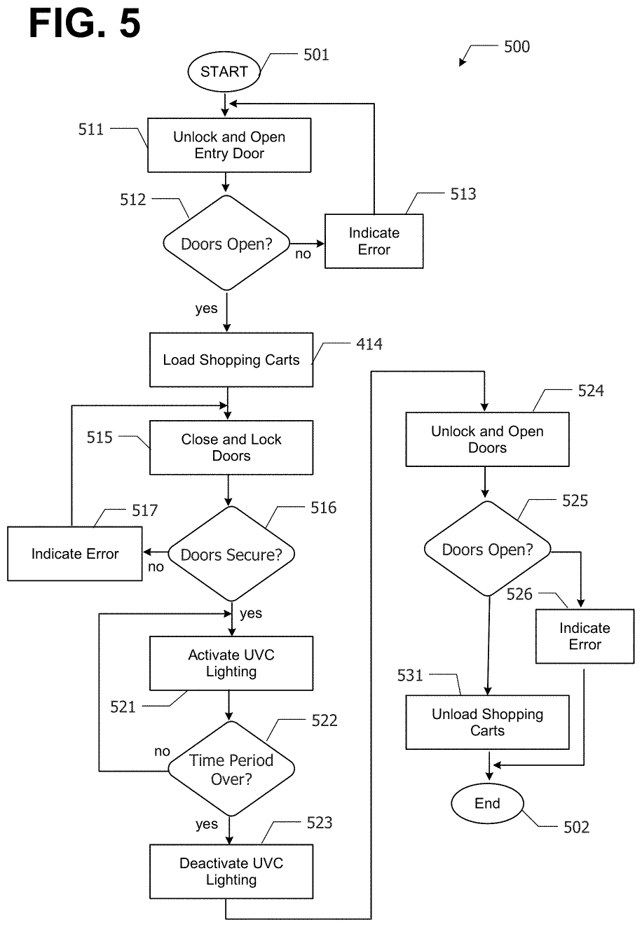

FIG. 5 illustrates a flowchart of a sequence of steps to implement a method for providing automated UVC cleaning of shopping carts according to the present invention. The process 500 starts 501 with the entrance door 201 being unlocked and opened in step 511. The controller timer 301 determines in test step 512 whether or not the entrance door 201 is open, and if not, step 512 provides an error indication and the process 500 returns to step 511 to try again.

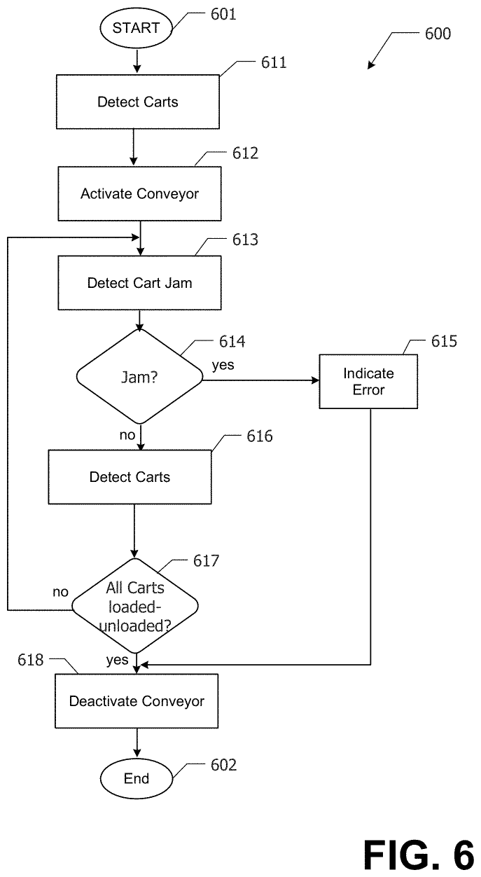

When test 512 determines that the entrance door 201 is open, the shopping carts 105a-c are loaded into the automated UVC cart cleaner 100 in step 514. The loading and unloading of shopping carts 105a-b performs the steps as shown in FIG. 6 described below. The entrance door opener 206a closes the entrance door 201 and the entrance door lock 205a is locked in step 515. Test step 516 determines whether or not the doors 201-202 are secure, and if not, an error indication is provided in step 517 and the process 500 returns to step 515 to try again.

When test step 516 determines that the doors 201-202 are secure, the UVC light source emitters 110 are activated in step 521 and test step 522 determines when the specified time period ends, at which time step 523 deactivates the UVC light source emitters 110. The door locks 205a-b are unlocked, and openers 206a-n activated to open the doors 201-202 in step 524. Test step 525 determines whether or not the doors 201-202 successfully opened, and if not, an error is indicated in step 526 before the process 500 ends 502. When test step 535 determines that the doors 201-202 were successfully opened, step 531 unloads the shopping carts 105a-c and the process 500 ends. As noted above, the loading and unloading of shopping carts 105a-b performs the steps as shown in FIG. 6 described below.

FIG. 6 illustrates a flowchart of a sequence of steps to implement a method for loading and uploading shopping carts by an automated UVC cleaning of shopping carts according to the present invention. The process 600 starts and shopping carts 105a-c are detected to be loaded or unloaded in step 611. The shopping carts 105a-c may be detected using optical contact sensors as discussed above. These optical contact sensors may be located at the entrance to the automated UVC cart cleaner 100, the exit from the automated UVC cart cleaner 100, and one or more locations within the automated UVC cart cleaner 100. The detection may also include optical contact sensors from each of multiple automated UVC cart cleaner units 100 in making the detection of the shopping carts 105a-c in embodiments having multiple connected units.

Next, step 612 activates the conveyor subsystem 207 before the controller-timer 301 begins detecting a shopping cart jam within the automated UVC cart cleaner 100 in step 613. Test step 614 causes the controller timer 301 to determine whether or not a jam exists, and if so, an error is indicated in step 615 and the conveyor subsystem 207 is deactivated and the process 600 ends. When test step 614 does not find a shopping cart jam, step 616 checks to see if shopping carts are still within the automated UVC cart cleaner 100. Test step 617 determines whether all of the shopping carts 105a-c are loaded or unloaded, and if not, the process returns to step 613; otherwise, the conveyor subsystem 207 is deactivated in step 618 and the process 600 ends 602.

Even though particular combinations of features are recited in the present application, these combinations are not intended to limit the disclosure of the invention. In fact, many of these features may be combined in ways not specifically recited in this application. In other words, any of the features mentioned in this application may be included to this new invention in any combination or combinations to allow the functionality required for the desired operations.

No element, act, or instruction used in the present application should be construed as critical or essential to the invention unless explicitly described as such. Further, the phrase “based on” is intended to mean “based, at least in part, on” unless explicitly stated otherwise.

What is claimed is:

1. A system for providing automated UVC cleaning of shopping carts, the system comprises:
a first enclosing tunnel for containing one or more shopping carts during cleaning;
one or more automatically opening and closing doors for enclosing any access to the first enclosing tunnel;
a door lock coupled to each of the automatically opening and closing doors;
a conveyor subsystem for loading and unloading shopping carts for cleaning within the first enclosing tunnel; and
one or more UVC light source emitters contained within the first enclosing tunnel.

2. The system according to claim 1, wherein the system further comprises:
a control panel for operating the system, the control panel comprises:
a controller-timer for activating the one or more automatically opening and closing doors, the door locks coupled to each of the one or more automatically opening and closing doors, the conveyor subsystem, and the UVC light source emitters;
an input keypad; and
an activating key for insertion into a control panel keyhole.

3. The system according to claim 2, wherein the controller-time comprise:
a memory having instructions stored thereon; and
a processor configured to execute the instructions on the memory to cause the control panel to:
receive an activation input when the activating key rotates within the control panel keyhole;
close the one or more automatically opening and closing doors;
activate the UVC light source emitters;
after the UVC light source emitters have been active for a predetermined length of time, deactivate the UVC light source emitters; and
open the one or more automatically opening and closing doors.

4. The system according to claim 3, wherein the processor is configured to execute the additional instructions to further cause the control panel to:
lock the door locks on the one or more automatically opening and closing doors after the doors have been closed;
activate the UVC light source emitters only when the door locks remain locked; and
unlock the door locks to open the one or more automatically opening and closing doors only when the UVC light source emitters are deactivated.

5. The system according to claim 4, wherein the processor is configured to execute the additional instructions to further cause the control panel to:
detect a presence of shopping carts within the enclosing tunnel;
determine whether the one or more automatically opening and closing doors are open;
unlock and open the one or more automatically opening and closing doors, when the one or more automatically opening and closing doors are closed;
activate the conveyor subsystem to push the shopping carts out of the enclosing tunnel when the shopping carts are detected and the one or more automatically opening and closing doors are open;
detect when a last shopping cart has exited the enclosing tunnel; and
deactivate the conveyor subsystem.

6. The system according to claim 5, wherein the processor is configured to execute the additional instructions to further cause the control panel to:
detect a jam of one or more shopping carts within the enclosing tunnel;
activate an alarm on the control panel; and
deactivate the conveyor subsystem.

7. The system according to claim 4, wherein the processor is configured to execute the additional instructions to further cause the control panel to:
detect a presence of shopping carts within the enclosing tunnel;
when no shopping carts are detected within the enclosing tunnel, perform the following steps:
detect a presence of shopping carts within an entrance to the enclosing tunnel;
determine whether the one or more automatically opening and closing doors are open;
unlock and open the one or more automatically opening and closing doors, when the one or more automatically opening and closing doors are closed;
activate the conveyor subsystem to push the shopping carts into the enclosing tunnel;
detect when a last shopping cart has exited the entrance to the enclosing tunnel;
deactivate the conveyor subsystem; and
close and lock the one or more automatically opening and closing doors.

8. The system according to claim 4, wherein the system further comprises:
a second enclosing tunnel coupled to the first enclosing tunnel;
a second conveyor subsystem for loading and unloading shopping carts for cleaning within the second enclosing tunnel, the second conveyor subsystem receives shopping carts from the first conveyor subsystem and pushes the shopping carts out of the second enclosing tunnel; and
one or more UVC light source emitters contained within the second enclosing tunnel.

9. The system according to claim 8, wherein the control panel:
activates the first conveyor subsystem and the second conveyor subsystem at the same time;
deactivates the first conveyor subsystem and the second conveyor subsystem at the same time;
activates the UVC light source emitters within the first enclosing tunnel and the UVC light source emitters within the second enclosing tunnel at the same time; and
deactivates the UVC light source emitters within the first enclosing tunnel and the UVC light source emitters within the second enclosing tunnel at the same time.

10. The system according to claim 4, wherein the system has only one automatically opening and closing door and the conveyor subsystem pushes the shopping carts into the enclosing tunnel and out of the enclosing tunnel using the same opening.

11. A method for providing automated UVC cleaner unit of shopping carts, the unit comprises a first enclosing tunnel for containing one or more shopping carts during cleaning, one or more automatically opening and closing doors for enclosing any access to the first enclosing tunnel, a door lock coupled to each of the automatically opening and closing doors, a conveyor subsystem for loading and unloading shopping carts for cleaning within the first enclosing tunnel, and one or more UVC light source emitters contained within the first enclosing tunnel, the method comprises:
receiving an activation input when the activating key rotates within the control panel keyhole;
closing the one or more automatically opening and closing doors;
activating the UVC light source emitters;
after the UVC light source emitters have been active for a predetermined length of time, deactivating the UVC light source emitters; and
opening the one or more automatically opening and closing doors.

12. The method according to claim 11, wherein the method further:
locking the door locks on the one or more automatically opening and closing doors after the doors have been closed;
activating the UVC light source emitters only when the door locks remain locked; and
unlocking the door locks to open the one or more automatically opening and closing doors only when the UVC light source emitters are deactivated.

13. The method according to claim 12, wherein the method further comprises:
detecting a presence of shopping carts within the enclosing tunnel;
determining whether the one or more automatically opening and closing doors are open;
unlocking and opening the door locks coupled to each of the one or more automatically opening and closing doors, when the one or more automatically opening and closing doors are closed;
activating the conveyor subsystem to push the shopping carts out of the enclosing tunnel when the shopping carts are detected and the one or more automatically opening and closing doors are open;
detecting when a last shopping cart has exited the enclosing tunnel; and
deactivating the conveyor subsystem.

14. The method according to claim 13, wherein the method further comprises:
detecting a jam of one or more shopping carts within the enclosing tunnel;
activating an alarm on the control panel; and
deactivating the conveyor subsystem.

15. The method according to claim 14, wherein method further comprises:
detecting a presence of shopping carts within the enclosing tunnel;
when no shopping carts are detected within the enclosing tunnel, performing the following steps:
detecting a presence of shopping carts within an entrance to the enclosing tunnel;
determining whether the one or more automatically opening and closing doors are open;
unlocking and opening the door locks coupled to each of the one or more automatically opening and closing doors, when the one or more automatically opening and closing doors are closed;
activating the conveyor subsystem to push the shopping carts into the enclosing tunnel;
detecting when a last shopping cart has exited the entrance to the enclosing tunnel;
deactivating the conveyor subsystem; and
closing and locking the door locks coupled to each of the one or more automatically opening and closing doors.
